(19) United States
(12) Patent Application Publication
(10) Pub. No.: US 2024/0225759 A1
(43) Pub. Date: Jul. 11, 2024

(54) ACTUATION ASSEMBLIES FOR SURGICAL INSTRUMENTS SUCH AS FOR USE IN ROBOTIC SURGICAL SYSTEMS

(71) Applicant: Covidien LP, Mansfield, MA (US)

(72) Inventor: Zachary S. Heiliger, Nederland, CO (US)

(21) Appl. No.: 18/561,625

(22) PCT Filed: May 9, 2022
(86) PCT No.: PCT/IB2022/054278

§ 371 Date: Nov. 16, 2023

Related U.S. Application Data
(60) Provisional application No. 63/190,288, filed on May 19, 2021.

(51) Int. Cl. A61B 34/30; A61B 34/00

(52) U.S. Cl. CPC A61B 34/30; A61B 34/71; A61B 2034/301

(57) ABSTRACT

An actuation assembly of a surgical instrument includes an actuation mechanism and an output mechanism. The actuation mechanism includes a piston assembly configured for positive and negative actuation. The output mechanism includes a fluid vessel in fluid communication with a fluid line connected to the piston assembly, a piston sealingly and slidably disposed within the fluid vessel, and a driver coupled to the piston. In response to positive actuation of the piston assembly, the pressurized fluid flows into the fluid vessel to move the piston to translate the driver in one direction. In response to negative actuation, the pressurized fluid exits the fluid vessel into the fluid line to allow the piston to translate the driver in an opposite direction.

### CROSS-REFERENCE TO RELATED APPLICATION

This application is a 371 National Stage Application of International Application No. PCT/IB2022/054278, filed May 9, 2022, which claims the benefit of, and priority to, U.S. Provisional Patent Application No. 63/190,288, filed on May 19, 2021, the entire contents of each of which are hereby incorporated herein by reference.

### FIELD

The present disclosure relates to surgical instruments and, more specifically, to actuation assemblies, e.g., including one or more actuation mechanisms each having one or more associated articulation mechanisms, drive actuation mechanisms, deployment actuation mechanisms, etc., for surgical instruments such as for use in robotic surgical systems.

### BACKGROUND

Robotic surgical systems are increasingly utilized in various different surgical procedures. Some robotic surgical systems include a console supporting a robotic arm. One or more different surgical instruments may be configured for use with the robotic surgical system and selectively mountable to the robotic arm. The robotic arm provides one or more inputs to the mounted surgical instrument to enable operation of the mounted surgical instrument.

### SUMMARY

As used herein, the term “distal” refers to the portion that is being described which is farther from an operator (whether a human surgeon or a surgical robot), while the term “proximal” refers to the portion that is being described which is closer to the operator. The terms “about,” substantially,” and the like, as utilized herein, are meant to account for manufacturing, material, environmental, use, and/or measurement tolerances and variations, and in any event may encompass differences of up to 10%. Further, to the extent consistent, any of the aspects described herein may be used in conjunction with any or all of the other aspects described herein.

Provided in accordance with aspects of the present disclosure is an actuation assembly of a surgical instrument. The actuation assembly includes an actuation mechanism and an output mechanism. The actuation mechanism includes a piston assembly configured such that positive actuation of the piston assembly urges pressurized fluid from the piston assembly along a fluid line and such that negative actuation of the piston assembly allows the pressurized fluid to return to the piston assembly from the fluid line. The output mechanism includes a fluid vessel defining a fluid port in fluid communication with the fluid line, a piston sealingly and slidably disposed within the fluid vessel, and a driver coupled to the piston. In response to positive actuation of the piston assembly, the pressurized fluid flows into the fluid-receiving portion of the fluid vessel to move the piston to thereby translate the driver in a first direction. In response to negative actuation of the piston assembly, the pressurized fluid exits the fluid-receiving portion of the fluid vessel into the fluid line to allow the piston to move to thereby translate the driver in a second, opposite direction.

In an aspect of the present disclosure, a biasing member is disposed within the fluid vessel to bias the piston. In such aspects, in response to positive actuation of the piston assembly, the pressurized fluid flows into the fluid-receiving portion of the fluid vessel to move the piston against the bias of the biasing member to thereby translate the driver in a first direction. In response to negative actuation of the piston assembly, the pressurized fluid exits the fluid-receiving portion of the fluid vessel into the fluid line and the biasing member moves the piston under the bias thereof to thereby translate the driver in a second, opposite direction.

In an aspect of the present disclosure, the actuation mechanism further includes a lead screw assembly configured to receive a rotational input and to convert the rotational input into longitudinal motion. The lead screw assembly is coupled to the piston assembly such that forward longitudinal motion produced by the lead screw assembly positively actuates the piston assembly and such that reverse longitudinal motion produced by the lead screw assembly negatively actuates the piston assembly.

In another aspect of the present disclosure, the lead screw assembly is configured to receive the rotational input from an input coupler configured to connect to a robotic surgical system.

In another aspect of the present disclosure, the lead screw assembly includes a lead screw coupled with the input coupler such that rotation of the input coupler rotates the lead screw in a similar manner. The lead screw assembly further includes a nut threadingly engaged about the lead screw such that rotation of the lead screw translates the nut along the lead screw to provide the longitudinal motion.

In still another aspect of the present disclosure, at least a portion of the fluid line is flexible to permit articulation thereof to enable articulation of the output mechanism relative to the actuation mechanism.

In yet another aspect of the present disclosure, a flexible cable extends through the fluid line and is coupled between a piston of the piston assembly and the piston of the output mechanism.

In still yet another aspect of the present disclosure, the driver is or is connected to a drive rod configured to actuate at least one of a first jaw member or a second jaw member relative to the other of the first jaw member or the second jaw member between a spaced-apart position and an approximated position for grasping tissue therebetween.

In another aspect of the present disclosure, the driver is or is connected to a knife support bar supporting a knife configured for translation relative to an end effector assembly between a retracted position and a deployed position to cut tissue grasped by the end effector assembly.

In yet another aspect of the present disclosure, the driver is or is connected to a first articulation cable configured to articulate an end effector assembly relative to a housing. In such aspects, the driver is connected to the first articulation cable and further connected to a second actuation cable. In such aspects, the first and second articulation cables are configured to move in opposite directions in response to translation of the driver.

A surgical instrument configured for attachment to a robotic surgical system and provided in accordance with the present disclosure includes a housing, a shaft extending distally from the housing and including an articulation section, an end effector assembly extending distally from the shaft such that the articulation section of the shaft is disposed between the housing and the end effector assembly, an actuation mechanism disposed within the housing, and an output mechanism disposed distally of the articulation section of the shaft. The actuation mechanism includes a piston assembly configured such that positive actuation of the piston assembly urges pressurized fluid from the piston assembly along a fluid line and such that negative actuation of the piston assembly allows the pressurized fluid to return to the piston assembly from the fluid line. The fluid line extends from the housing and at least partially through the shaft including the articulation section thereof. The output mechanism includes a fluid vessel defining a fluid port in fluid communication with the fluid line, a piston sealingly and slidably disposed within the fluid vessel, and a driver coupled to the piston and configured to actuate the end effector assembly or a component relative to the end effector assembly. In response to positive actuation of the piston assembly, the pressurized fluid flows into the fluid vessel to move the piston to thereby translate the driver in a first direction. In response to negative actuation of the piston assembly, the pressurized fluid exits the fluid vessel into the fluid line to allow the piston to move to thereby translate the driver in a second, opposite direction.

In an aspect of the present disclosure, a biasing member is disposed within the fluid vessel to bias the piston. In such aspects, in response to positive actuation of the piston assembly, the pressurized fluid flows into the fluid-receiving portion of the fluid vessel to move the piston against the bias of the biasing member to thereby translate the driver in a first direction. In response to negative actuation of the piston assembly, the pressurized fluid exits the fluid-receiving portion of the fluid vessel into the fluid line and the biasing member moves the piston under the bias thereof to thereby translate the driver in a second, opposite direction.

In an aspect of the present disclosure, at least a portion of the fluid line is flexible to permit articulation thereof upon articulation of the articulation section of the shaft.

In another aspect of the present disclosure, a flexible cable extends through the fluid line and is coupled between a piston of the piston assembly and the piston of the output mechanism. In still another aspect of the present disclosure, the driver is or is connected to a drive rod configured to actuate at least one of a first jaw member or a second jaw member of the end effector assembly relative to the other of the first jaw member or the second jaw member between a spaced-apart position and an approximated position for grasping tissue therebetween.

In yet another aspect of the present disclosure, the driver is or is connected to a knife support bar supporting a knife. The knife is the component and is configured for translation relative to the end effector assembly between a retracted position and a deployed position to cut tissue grasped by the end effector assembly.

Another surgical instrument configured for attachment to a robotic surgical system and provided in accordance with the present disclosure includes a housing, a shaft extending distally from the housing and having an articulation section, an end effector assembly extending distally from the shaft such that the articulation section of the shaft is disposed between the housing and the end effector assembly, at least one articulation cable extending though the articulation section such that actuation of the at least one articulation cable articulates the articulation section to thereby articulate the end effector assembly relative to the housing, an actuation mechanism disposed within the housing, and an output mechanism disposed distally of the articulation section of the shaft. The actuation mechanism includes a piston assembly configured such that positive actuation of the piston assembly urges pressurized fluid from the piston assembly along a fluid line and such that negative actuation of the piston assembly allows the pressurized fluid to return to the piston assembly from the fluid line. The fluid line extends from the housing and at least partially through the shaft including the articulation section thereof. The output mechanism includes a fluid vessel defining a fluid port in fluid communication with the fluid line, a piston sealingly and slidably disposed within the fluid vessel, and a biasing member disposed within the fluid vessel to bias the piston. The at least one articulation cable is coupled to the piston. In response to positive actuation of the piston assembly, the pressurized fluid flows into the fluid vessel to move the piston against the bias of the biasing member to thereby translate the at least one articulation cable in a first direction. In response to negative actuation of the piston assembly, the pressurized fluid exits the fluid vessel into the fluid line under the bias of the biasing member moving the piston to thereby translate the at least one articulation cable in a second, opposite direction.

In an aspect of the present disclosure, the actuation mechanism further includes a lead screw assembly configured to receive a rotational input and to convert the rotational input into longitudinal motion. In such aspects, the lead screw assembly may be coupled to the piston assembly such that forward longitudinal motion produced by the lead screw assembly positively actuates the piston assembly and such that reverse longitudinal motion produced by the lead screw assembly negatively actuates the piston assembly.

In another aspect of the present disclosure, the surgical instrument further includes an input coupler configured to connect to a robotic surgical system. In such aspects, the lead screw assembly is configured to receive the rotational input from the input coupler.

In still another aspect of the present disclosure, the lead screw assembly includes a lead screw coupled with the input coupler such that rotation of the input coupler rotates the lead screw in a similar manner. A nut is threadingly engaged about the lead screw such that rotation of the lead screw translates the nut along the lead screw to provide the longitudinal motion.

In yet another aspect of the present disclosure, the at least one articulation cable includes two articulation cables and wherein actuation of the piston assembly translates the two articulation cables in opposite directions.

### BRIEF DESCRIPTION OF THE DRAWINGS

Various aspects and features of the present disclosure are described hereinbelow with reference to the drawings wherein:

FIG. 1 is a perspective view of a surgical instrument in accordance with the present disclosure configured for mounting on a robotic arm of a robotic surgical system;

FIG. 2 is a perspective view of a proximal end portion of the surgical instrument of FIG. 1 with portions removed;

FIG. 3A is a first side view illustrating first and third actuation mechanisms of the surgical instrument of FIG. 1;

FIG. 3B is a second side view illustrating second and fourth actuation mechanisms of the surgical instrument of FIG. 1;

FIG. 4 is a schematic illustration of an exemplary robotic surgical system configured to releasably receive the surgical instrument of FIG. 1;

FIG. 5 is a side, partial longitudinal cross-sectional view illustrating the third and fourth actuation mechanisms operably coupled to the cutting drive mechanism and the jaw drive mechanism, respectively, of the end effector assembly of the surgical instrument of FIG. 1;

FIG. 6 is a partial longitudinal cross-sectional view of the end effector assembly, the cutting drive mechanism, and the jaw drive mechanism of the surgical instrument of FIG. 1;

FIG. 7 is a side, partial longitudinal cross-sectional view of the fourth actuation mechanism operably coupled to the jaw drive mechanism of the surgical instrument of FIG. 1;

FIG. 8 is a side, partial longitudinal cross-sectional view of the third actuation mechanism operably coupled to the cutting drive mechanism of the surgical instrument of FIG. 1;

FIG. 9 is a side, partial longitudinal cross-sectional view illustrating the first actuation mechanism operably coupled to first and second articulation mechanisms, respectively, including respective first and second articulation cables extending through the articulation section of the surgical instrument of FIG. 1 to the end effector assembly thereof;

FIG. 10 is a side, partial longitudinal cross-sectional view illustrating the second actuation mechanism operably coupled to third and fourth articulation mechanisms, respectively, including respective first and second articulation cables extending therefrom; and

FIG. 11 is a side, partial longitudinal cross-sectional view illustrating another articulation mechanism in accordance with the present disclosure operably coupled to an actuation mechanism and including first and second articulation cables extending therefrom.

### DETAILED DESCRIPTION

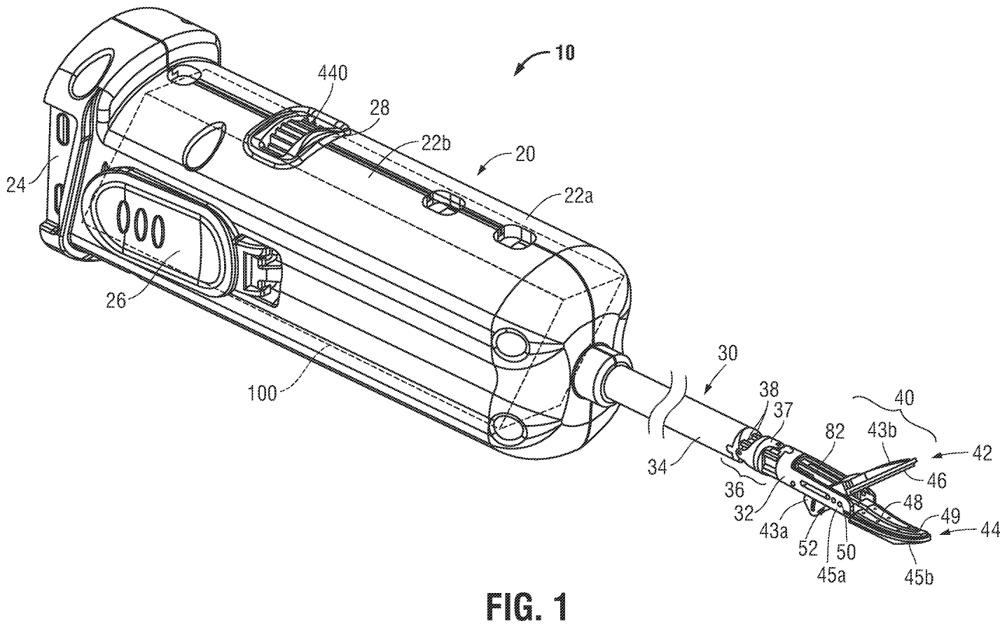

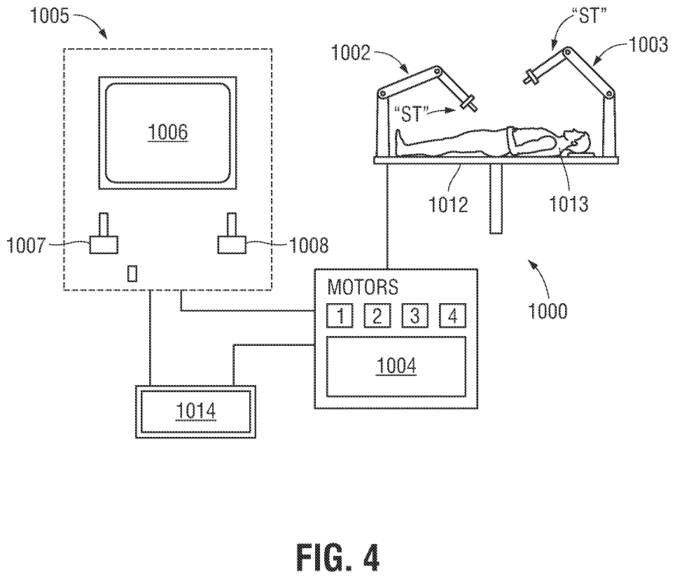

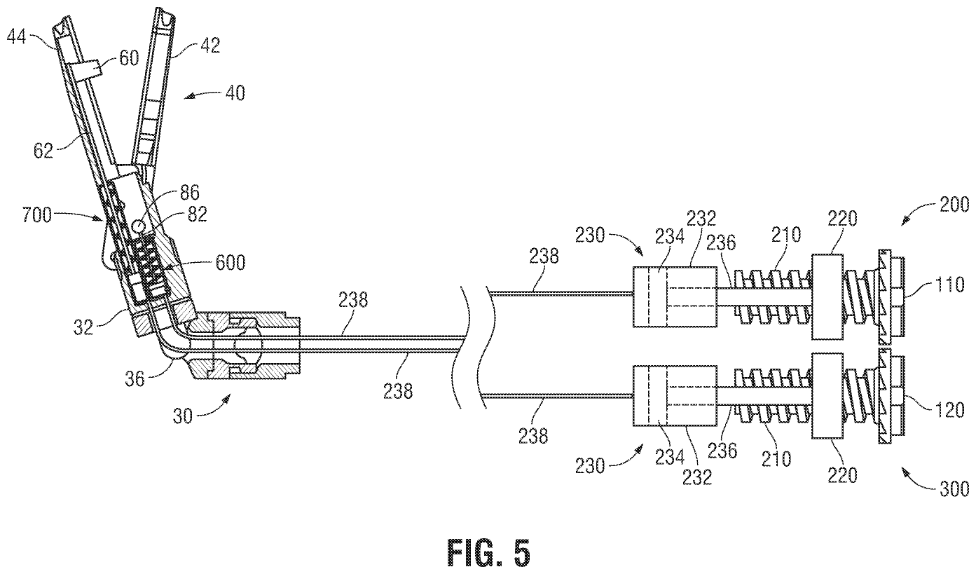

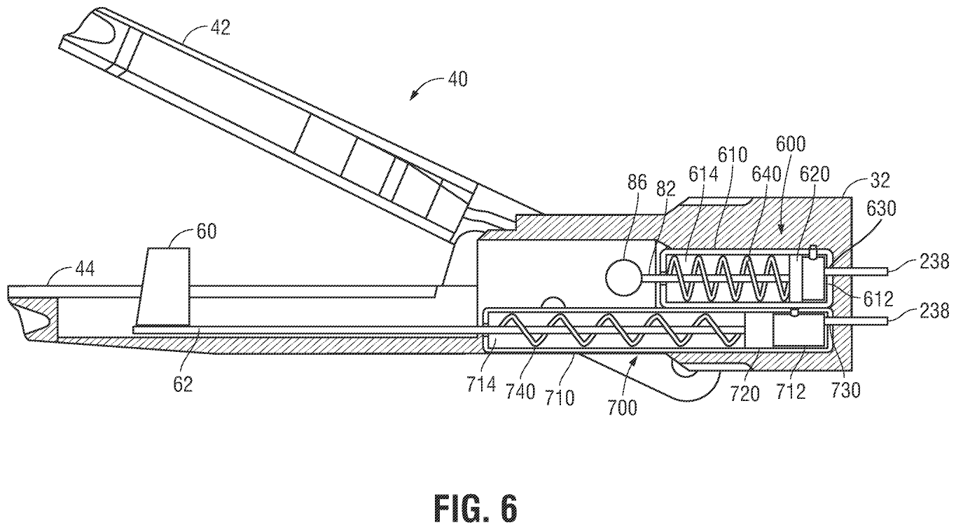

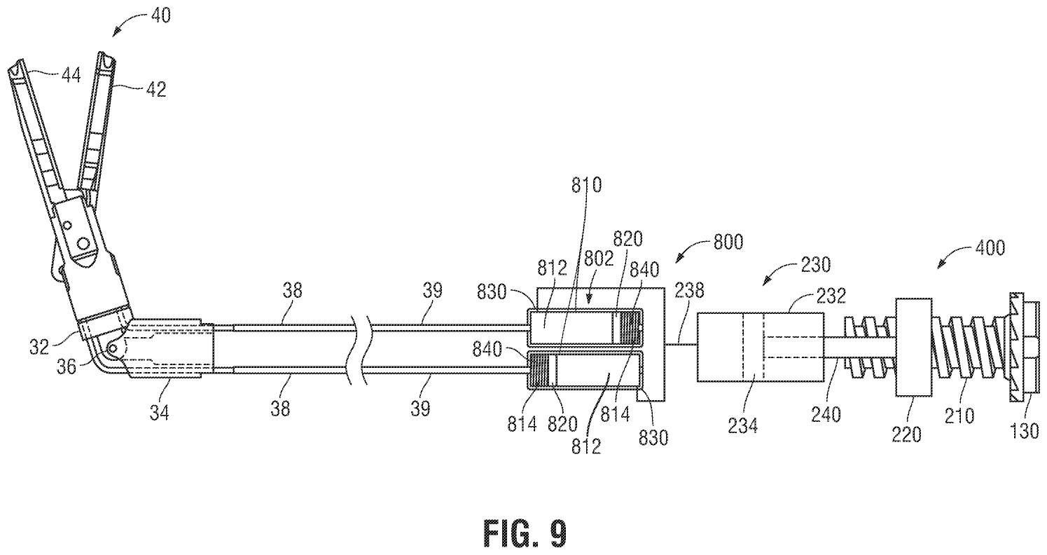

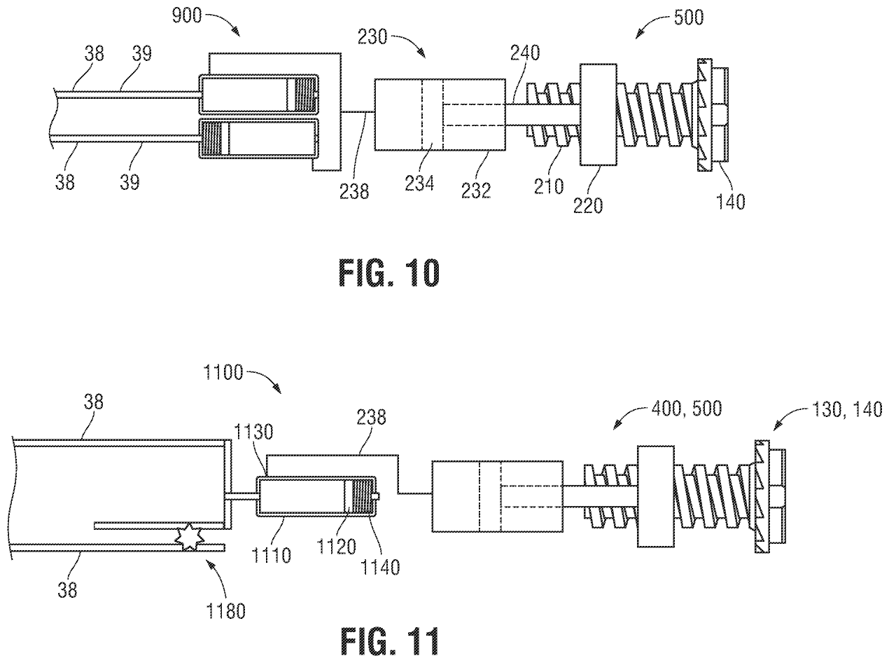

Referring to FIGS. 1-3B, a surgical instrument 10 provided in accordance with the present disclosure generally includes a housing 20, a shaft 30 extending distally from housing 20, an end effector assembly 40 extending distally from shaft 30, and an actuation assembly 100. Actuation assembly 100 includes a plurality of actuation mechanisms 200, 300, 400, 500 (FIGS. 3A and 3B), a jaw drive mechanism 600 (FIGS. 5-7) operably coupled to actuation mechanism 200, a cutting drive mechanism 700 (FIGS. 5, 6, and 8) operably coupled to actuation mechanism 300, and first and second articulation mechanisms 800, 900 (FIGS. 9 and 10, respectively) operably coupled to actuation mechanisms 400, 500, respectively. Instrument 10 is detailed herein as an articulating electrosurgical forceps including a deployable knife 60 (FIG. 6) that is configured for use with a robotic surgical system, e.g., robotic surgical system 1000 (FIG. 4). However, the aspects and features of instrument 10 provided in accordance with the present disclosure, detailed below, are equally applicable for use with other suitable surgical instruments, e.g., graspers, staplers, clip appliers; with other energy modalities, e.g., ultrasonic, microwave, thermal, etc.; for actuating other components and/or functions; and/or in other suitable surgical systems, e.g., motorized handheld or manual handheld systems.

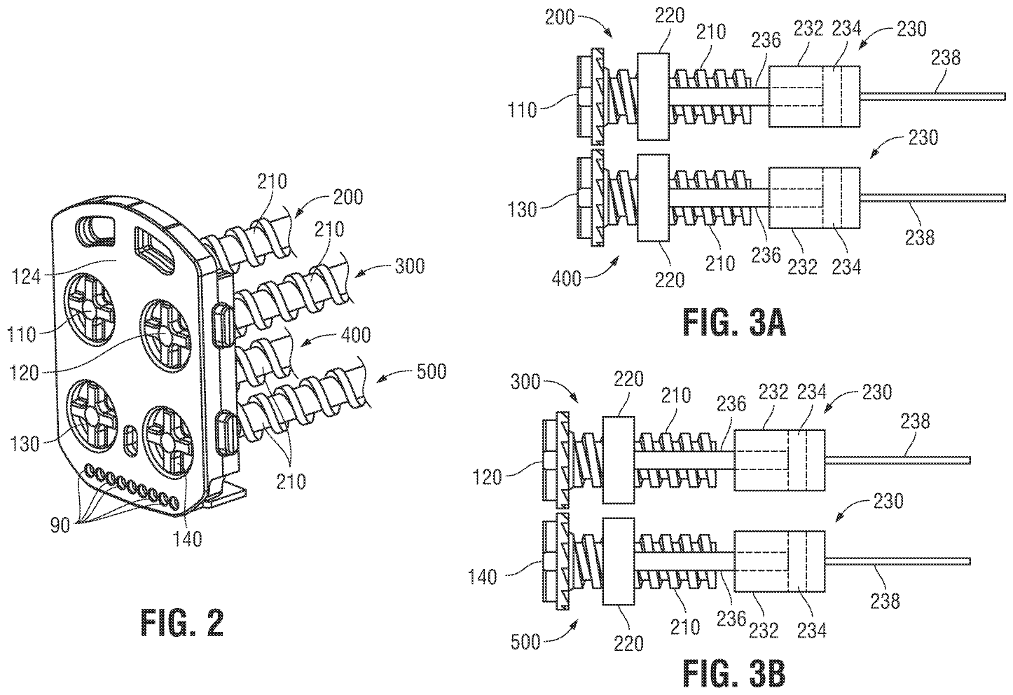

With reference to FIG. 1, housing 20 of instrument 10 includes first and second body portions 22a, 22b and a proximal face plate 24 that cooperate to enclose actuation assembly 100 and first and second articulation assemblies 800, 900 (FIGS. 9 and 10). Proximal face plate 24 includes apertures defined therein through which input couplers 110-140 (FIGS. 2 and 3A-3B) extend. A pair of latch levers 26 (only one of which is illustrated in FIG. 1) extending outwardly from opposing sides of housing 20 enable releasable engagement of housing 20 with a robotic arm of a surgical system, e.g., robotic surgical system 1000 (FIG. 4). An aperture 28 defined through housing 20 permits thumbwheel 440 to extend therethrough to enable manual manipulation of thumbwheel 440 from the exterior of housing 20 to permit manual opening and closing of end effector assembly 40. Thumbwheel 440 is operably coupled or couplable, for example, to lead screw 210 (FIG. 3B) to enable manual rotation thereof, e.g., in place of the motorized rotation provided by robotic surgical system 1000 (FIG. 4).

Referring also to FIG. 2, a plurality of electrical contacts 90 extend through one or more apertures defined through proximal face plate 24 to enable electrical communication between instrument 10 and robotic surgical system 1000 (FIG. 4) when instrument 10 is engaged thereon, e.g., for the communication of data, control, and/or power signals therebetween. As an alternative to electrical contacts 90 extending through proximal face plate 24, other suitable transmitter, receiver, and/or transceiver components to enable the communication of data, control, and/or power signals are also contemplated, e.g., using RFID, Bluetooth®, WiFi®, or via any other suitable wired, wireless, contacted, or contactless communication method. At least some of the electrical contacts 90 are electrically coupled with electronics (not shown) mounted on an interior side of proximal face plate 24, e.g., within housing 20. The electronics may include, for example, a storage device, a communications device (including suitable input/output components), and a CPU including a memory and a processor. The electronics may be mounted on a circuit board or otherwise configured, e.g., as a chip. The storage device of the electronics stores information relating to surgical instrument 10 such as, for example: the item number, e.g., SKU number; date of manufacture; manufacture location, e.g., location code; serial number; lot number; use information; setting information; adjustment information; calibration information; security information, e.g., encryption key(s), and/or other suitable additional or alternative data. The storage device may be, for example, a magnetic disk, flash memory, optical disk, or other suitable data storage device.

As an alternative or in addition to storing the above-noted information in the storage device of the electronics, some or all of such information, e.g., the use information, calibration information, setting information, and/or adjustment information, may be stored in a storage device associated with robotic surgical system 1000 (FIG. 4), a remote server, a cloud server, etc., and accessible via instrument 10 and/or robotic surgical system 1000 (FIG. 4). In such configurations, the information may, for example, be updated by manufacturer-provided updates, and/or may be applied to individual instruments, units of instruments (e.g., units from the same manufacturing location, manufacturing period, lot number, etc.), or across all instruments. Further still, even where the information is stored locally on each instrument, this information may be updated by manufacturer-provided updates manually or automatically upon connection to the robotic surgical system 1000 (FIG. 4).

With reference to FIGS. 2 and 3A-3B, input couplers 110, 120, 130, 140 are rotatably disposed within proximal face plate 24 and configured to receive rotational input from robotic surgical system 1000 (FIG. 4) when surgical instrument 10 (FIG. 1) is mounted on robotic surgical system 1000 (FIG. 4). Input couplers 110, 120, 130, 140, in turn, are operably coupled with actuation mechanisms 200, 300, 400, 500, respectively. More specifically, each actuation mechanism 200, 300, 400, 500 includes a lead screw 210 engaged in fixed rotational relation with a respective input coupler 110, 120, 130, 140 such that rotation of an input coupler 110, 120, 130, 140 effects similar rotation of a corresponding lead screw 210. A nut 220 is threadingly disposed about each lead screw 210 such that rotation of a lead screw 210 translates the corresponding nut 220 therealong. Thus, rotational input(s) provided by robotic surgical system 1000 (FIG. 4) are converted into longitudinal translation of nut(s) 220 in either direction based upon the direction of the rotational input. Nuts 220, in turn, are operably coupled, e.g., fixedly engaged or otherwise coupled, with piston assemblies 230.

Piston assemblies 230 include fluid vessels 232, piston heads 234 scalingly and slidably disposed within fluid vessels 232, and piston rods 236 engaged with piston heads 234. Nuts 220 are operably coupled with piston heads 234 of piston assemblies 230 such that translation of nuts 220 urges piston heads 234 through the corresponding fluid vessels 232. Fluid vessels 232 retain a pressurized fluid, e.g., air, other gas, water, saline, other liquid, etc., such that sliding of piston heads 234 within fluid vessels 232 in a first direction drives pressurized fluid out through fluid lines 238 and such that that sliding of piston heads 234 within fluid vessels 232 in a second, opposite direction draws (or enables the flow of) pressurized fluid from fluid lines 238 back into fluid vessels 232.

The four piston assemblies 230 are operably coupled to jaw drive mechanism 600 (FIGS. 5-7), cutting drive mechanism 700 (FIGS. 5, 6, and 8), first articulation mechanism 800 (FIG. 9), and second articulation mechanism 900 (FIG. 10), respectively, to enable fluid-driven actuation thereof. More specifically, as detailed below, actuation of actuation mechanisms 200, 300, 400, 500 (e.g., in response to rotational inputs provided by robotic surgical system 1000 (FIG. 4)) provides fluid-driven opening and closing of end effector assembly 40, deployment and retraction of knife 60 (FIG. 6), and pitch and/or yaw articulation of end effector assembly 40 relative to shaft 30.

Returning to FIG. 1, shaft 30 of instrument 10 includes a distal segment 32, a proximal segment 34, and an articulating section 36 disposed between the distal and proximal segments 32, 34, respectively. Articulating section 36 includes one or more articulating components 37, e.g., links, joints, etc. A plurality of articulation cables 38, e.g., four (4) articulation cables, or other suitable actuators, extend through articulating section 36. More specifically, articulation cables 38 are operably coupled to distal segment 32 of shaft 30 at the distal ends thereof and extend proximally from distal segment 32 of shaft 30, through articulating section 36 of shaft 30 and proximal segment 34 of shaft 30, and into housing 20, wherein first and second pairs of the articulation cables 38 operably couple with respective articulation mechanisms 800, 900 (FIGS. 9 and 10) to enable selective articulation of distal segment 32 (and, thus end effector assembly 40) relative to proximal segment 34 and housing 20, e.g., about at least two axes of articulation (yaw and pitch articulation, for example). Articulation cables 38 are arranged in a generally rectangular configuration, although other suitable configurations are also contemplated. In some configurations, as an alternative, shaft 30 is substantially rigid, malleable, or flexible and not configured for active articulation.

With respect to articulation of end effector assembly 40 relative to proximal segment 34 of shaft 30, actuation of articulation cables 38 may be accomplished in pairs. More specifically, in order to pitch end effector assembly 40, the upper pair of cables 38 are actuated in a similar manner while the lower pair of cables 38 are actuated in a similar manner relative to one another but an opposite manner relative to the upper pair of cables 38. With respect to yaw articulation, the right pair of cables 38 are actuated in a similar manner while the left pair of cables 38 are actuated in a similar manner relative to one another but an opposite manner relative to the right pair of cables 38. Thus, whether pitch and/or yaw articulation is actuated, diagonally-opposed articulation cables 38 are always actuated in opposite manners. Thus, only two articulation mechanisms 800, 900 (FIGS. 9 and 10) are required to enable any combination of pitch and/or yaw articulation: a first articulation mechanism 800 (FIG. 9) that actuates a first pair of diagonally-opposed articulation cables 38 in equal magnitude but opposite directions; and a second articulation mechanism 900 (FIG. 10) that actuates a second pair of diagonally-opposed articulation cables 38 in equal magnitude but opposite directions. Other configurations of articulation cables 38 are also contemplated.

Continuing with reference to FIG. 1, end effector assembly 40 includes first and second jaw members 42, 44, respectively. Each jaw member 42, 44 includes a proximal flange portion 43a, 45a and a distal body portion 43b, 45b, respectively. Distal body portions 43b, 45b define opposed tissue-contacting surfaces 46, 48, respectively. Proximal flange portions 43a, 45a are pivotably coupled to one another about a pivot 50 and are operably coupled to one another via a cam-slot assembly 52 including a cam pin 86 (FIGS. 5-7) slidably received within cam slots defined within the proximal flange portion 43a, 45a of at least one of the jaw members 42, 44, respectively, to enable pivoting of jaw member 42 relative to jaw member 44 and distal segment 32 of shaft 30 between a spaced-apart position (e.g., an open position of end effector assembly 40) and an approximated position (e.g., a closed position of end effector assembly 40) for grasping tissue between tissue-contacting surfaces 46, 48. As an alternative to this unilateral configuration, a bilateral configuration may be provided whereby both jaw members 42, 44 are pivotable relative to one another and distal segment 32 of shaft 30. Other suitable configurations are also contemplated. In aspects, one or more stop structures are provided on, extending through, or otherwise associated with either or both tissue-contacting surfaces 46, 48 to maintain a minimum gap distance between tissue-contacting surfaces 46, 48 in the approximated position thereof.

In configurations, a longitudinally-extending knife channel 49 (only knife channel 49 of jaw member 44 is illustrated; the knife channel of jaw member 42 is similarly configured) is defined through the tissue-contacting surface 46, 48 of one or both jaw members 42, 44. With additional reference to FIG. 6, a knife 60 supported on a knife support rod 62 is selectively translatable through the knife channel(s) 49 and between the jaw members 42, 44 to cut tissue grasped between tissue-contacting surfaces 46, 48 of jaw members 42, 44, respectively. Knife support rod 62 is operably coupled to a cutting drive mechanism 700 (FIGS. 5, 6, and 8) which, in turn, is operably coupled to actuation mechanism 300 to enable the selective actuation of knife support rod 62 to, in turn, reciprocate knife 60 between jaw members 42, 44 to cut tissue grasped between tissue-contacting surfaces 46, 48. As an alternative to a longitudinally-movable mechanical knife, other suitable mechanical cutters are also contemplated, e.g., guillotine-style cutters, as are energy-based cutters, e.g., RF electrical cutters, ultrasonic cutters, etc., in static or dynamic configurations.

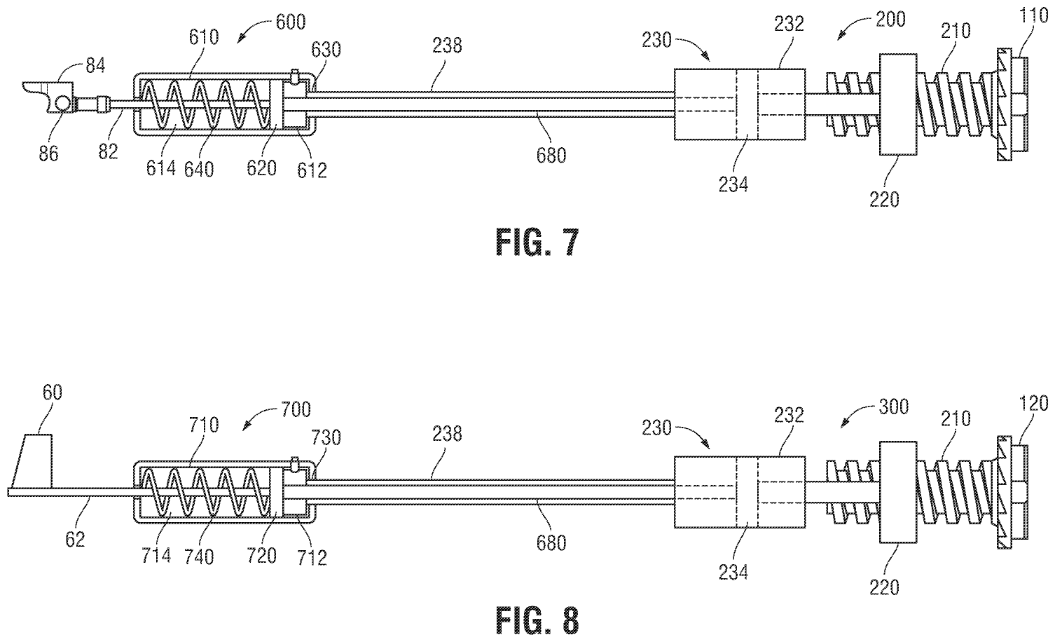

Referring still to FIG. 1, a drive rod 82 supporting a cam block 84 (FIG. 7) having a cam pin 86 (FIG. 7) extending transversely from either side thereof is operably coupled to cam-slot assembly 52 of end effector assembly 40, e.g., cam pin 86 (FIG. 7) is engaged within cam slots associated with either or both jaw members 42, 44 such that longitudinal actuation of drive rod 82 pivots jaw member 42 relative to jaw member 44 between the spaced-apart and approximated positions. Drive rod 82, in turn, is operably coupled to jaw drive mechanism 600 (FIGS. 5-7) which, in turn, is operably coupled to actuation mechanism 200 to enable the selective actuation of drive rod 82 to thereby pivot jaw member 42 relative to jaw member 44 between the spaced-apart and approximated positions.

Tissue-contacting surfaces 46, 48 of jaw members 42, 44, respectively, are at least partially formed from an electrically conductive material and are energizable to different potentials to enable the conduction of RF electrical energy through tissue grasped therebetween, although tissue-contacting surfaces 46, 48 may alternatively be configured to supply any suitable energy, e.g., thermal, microwave, light, ultrasonic, ultrasound, etc., through tissue grasped therebetween for energy-based tissue treatment. Instrument 10 defines a conductive pathway (not shown) through housing 20 and shaft 30 to end effector assembly 40 that may include lead wires, contacts, and/or electrically-conductive components to enable electrical connection of tissue-contacting surfaces 46, 48 of jaw members 42, 44, respectively, to an energy source (not shown), e.g., an electrosurgical generator, for supplying energy to tissue-contacting surfaces 46, 48 to treat, e.g., seal, tissue grasped between tissue-contacting surfaces 46, 48.

Actuation assembly 100 is configured to operably interface with a robotic surgical system 1000 (FIG. 4) via input couplers 110-120 (FIG. 2) when instrument 10 is mounted on robotic surgical system 1000 (FIG. 4), to enable robotic operation of actuation assembly 100 to provide the above-detailed functionality. That is, robotic surgical system 1000 (FIG. 4) selectively provides inputs, e.g., rotational inputs to input couplers 110-140 (FIG. 2) to articulate end effector assembly 40, manipulate one or both of jaw members 42, 44 to grasp tissue therebetween, and/or advance knife 60 to cut tissue grasped between jaw members 42, 44. However, it is also contemplated that actuation assembly 100 be configured to interface with any other suitable surgical system, e.g., a manual surgical handle, a powered surgical handle, etc. For the purposes herein, robotic surgical system 1000 (FIG. 4) is generally described.

Turning to FIG. 4, robotic surgical system 1000 is configured for use in accordance with the present disclosure. Aspects and features of robotic surgical system 1000 not germane to the understanding of the present disclosure are omitted to avoid obscuring the aspects and features of the present disclosure in unnecessary detail.

Robotic surgical system 1000 generally includes a plurality of robot arms 1002, 1003; a control device 1004; and an operating console 1005 coupled with control device 1004. Operating console 1005 may include a display device 1006, which may be set up in particular to display three-dimensional images; and manual input devices 1007, 1008, by means of which a person, e.g., a surgeon, may be able to telemanipulate robot arms 1002, 1003 in a first operating mode. Robotic surgical system 1000 may be configured for use on a patient 1013 lying on a patient table 1012 to be treated in a minimally invasive manner. Robotic surgical system 1000 may further include a database 1014, in particular coupled to control device 1004, in which are stored, for example, pre-operative data from patient 1013 and/or anatomical atlases.

Each of the robot arms 1002, 1003 may include a plurality of members, which are connected through joints, and a mounted device which may be, for example, a surgical tool “ST.” One or more of the surgical tools “ST” may be instrument 10 (FIG. 1), thus providing such functionality on a robotic surgical system 1000.

Robot arms 1002, 1003 may be driven by electric drives, e.g., motors, connected to control device 1004. The motors, for example, may be rotational drive motors configured to provide rotational inputs, e.g., to selectively rotationally drive input couplers 110-140 (FIG. 2) of surgical instrument (FIG. 1) to accomplish a desired task or tasks. Control device 1004, e.g., a computer, may be configured to activate the motors, in particular by means of a computer program, in such a way that robot arms 1002, 1003, and, thus, their mounted surgical tools “ST” execute a desired movement and/or function according to a corresponding input from manual input devices 1007, 1008, respectively. Control device 1004 may also be configured in such a way that it regulates the movement of robot arms 1002, 1003 and/or of the motors.

Control device 1004, more specifically, may control one or more of the motors based on rotation, e.g., controlling to rotational position using a rotational position encoder (or Hall effect sensors or other suitable rotational position detectors) associated with the motor to determine a degree of rotation output from the motor and, thus, the degree of rotational input provided to the corresponding input coupler 110-140 (FIG. 2) of surgical instrument 10 (FIG. 1). Alternatively or additionally, control device 1004 may control one or more of the motors based on torque, current, or in any other suitable manner.

With reference to FIGS. 5-7, jaw drive mechanism 600 and actuation thereof via actuation mechanism 200 to effect opening and closing of end effector assembly 40 are described. As noted above, in response to rotational input provided by robotic surgical system 1000 (FIG. 4) to input coupler 110, pressurized fluid is urged distally through fluid line 238 or pulled (or allowed to return) proximally through fluid line 238, depending upon the direction of the rotational input. Fluid line 238, more specifically, defines an elongated, flexible configuration enabling fluid line 238 to extend from piston assembly 230 of actuation mechanism 200 within housing 20 (FIG. 1), through shaft 30 (FIG. 1) including articulation section 36 (FIG. 1) thereof, to distal section 32 of shaft 30 or end effector assembly 40 to connect to jaw drive mechanism 600. Jaw drive mechanism 600 may be disposed within end effector assembly 40 or distal section 32 of shaft 30 (or otherwise positioned distally of articulation section 36 of shaft 30 (see FIG. 1)) and includes a fluid vessel 610, a piston head 620 sealingly and slidably disposed within fluid vessel 610 to divide fluid vessel 610 into a fluid-receiving portion 612 and an opposing portion 614, a fluid port 630 fluidly coupling fluid-receiving portion 612 of fluid vessel 610 with fluid line 238 of piston assembly 230 of actuation mechanism 200, and a biasing member 640, e.g., a coil spring, disposed within opposing portion 614 of fluid vessel 610 and configured to bias piston head 620 towards fluid-receiving portion 612 of fluid vessel 610. A proximal portion of drive rod 82 is coupled to piston head 620 such that translation of piston head 620 within fluid vessel 610 likewise translates drive rod 82 in a similar manner. Drive rod 82, as noted above, supports, at a distal portion thereof, cam block 84 including cam pin 86 which is operably coupled to cam-slot assembly 52 of end effector assembly 40 (FIG. 1) such that longitudinal actuation of drive rod 82 pivots jaw member 42 relative to jaw member 44 between the spaced-apart and approximated positions. In aspects, a flexible cable 680 extends between and engages piston head 620 of jaw drive mechanism 600 with piston head 234 of actuation mechanism 200 to provide support and facilitate concomitant movement therebetween.

As a result of the above-detailed configuration, sliding of piston head 234 within fluid vessel 232 in the first direction urges pressurized fluid from fluid vessel 232 through fluid line 238 and fluid port 630 into fluid-receiving portion 612 of fluid vessel 610 whereby the pressurized fluid urges piston head 620 within fluid vessel 610 towards opposing portion 614 of fluid vessel 610 and against the bias of biasing member 640. This translation of piston head 620, in turn, translates drive rod 82 to thereby pivot jaw member 42 relative to jaw member 44 from the spaced-apart towards the approximated position, e.g., to grasp tissue therebetween.

In aspects, jaw clamping pressure, e.g., the pressure applied to tissue grasped between jaw member 42 and jaw member 44, may be regulated, e.g., to within a range of between and including about 3 kg/cm² to about 16 kg/cm² (as an average taken at at least proximal, middle, and distal locations along the length of tissue-contacting surfaces 46, 48 in the approximated position), via the pressurized fluid and/or biasing member 640. Thus, the pressurized fluid and/or biasing member 640 function as a force-limiting feature to enable regulation of the jaw clamping pressure applied to tissue grasped between jaw member 42 and jaw member 44.

In order to enable return of jaw members 42, 44 towards the spaced-apart position, an opposite rotational input to input coupler 110 is provided by robotic surgical system 1000 (FIG. 4) such that piston head 234 is slid within fluid vessel 232 in the second, opposite direction, enlarging the volume within fluid vessel 232 that can be occupied by the pressurized fluid. As a result, pressurized fluid may be urged and/or drawn into fluid vessel 232. More specifically, as the volume within fluid vessel 232 that can be occupied by the pressurized fluid is increased, the force exerted by the pressurized fluid on piston head 620 against the bias of biasing member 640 is reduced or eliminated such that biasing member 640 urges piston head 620 back towards fluid-receiving portion 612 of fluid vessel 610 to thereby urge the pressurized fluid out port 630, through fluid line 238, and into fluid vessel 232 until the pressurized fluid substantially fills the enlarged volume within fluid vessel 232. This movement of piston head 620 pulls drive rod 82 in the opposite direction as detailed above to thereby pivot jaw member 42 relative to jaw member 44 from the approximated position back towards the spaced-apart position.

Turning to FIGS. 5, 6, and 8, cutting drive mechanism 700 and actuation thereof via actuation mechanism 300 to deploy and retract knife 60 relative to end effector assembly 40 are described. As noted above, in response to rotational input provided by robotic surgical system 1000 (FIG. 4) to input coupler 120, pressurized fluid is urged distally through fluid line 238 or pulled (or allowed to return) proximally through fluid line 238 of actuation mechanism 300, depending upon the direction of the rotational input. Cutting drive mechanism 700 may be disposed within end effector assembly 40 or distal section 32 of shaft 30 (or otherwise positioned distally of articulation section 36 of shaft 30 (see FIG. 1)) and includes a fluid vessel 710, a piston head 720 sealingly and slidably disposed within fluid vessel 710 to divide fluid vessel 710 into a fluid-receiving portion 712 and an opposing portion 714, a fluid port 730 fluidly coupling fluid-receiving portion 712 of fluid vessel 710 with fluid line 238 of piston assembly 230 of actuation mechanism 300, and a biasing member 740, e.g., a coil spring, disposed within opposing portion 714 of fluid vessel 710 and configured to bias piston head 720 towards fluid-receiving portion 712 of fluid vessel 710. A proximal portion of knife support rod 62 is coupled to piston head 720 such that translation of piston head 720 within fluid vessel 710 likewise translates knife support rod 62 in a similar manner. Knife support rod 62, as noted above, supports, at a distal portion thereof knife 60 such that longitudinal actuation of knife support rod 62 deploys knife 60 to translate between jaw members 42, 44 or retracts knife 60 proximally from between jaw members 42, 44, depending upon the direction of actuation. In aspects, a flexible cable 680 extends between and engages piston head 720 of cutting drive mechanism 700 with piston head 234 of actuation mechanism 300 to provide support and facilitate concomitant movement therebetween.

In use, actuation mechanism 300 and cutting drive mechanism 700 operate in a similar manner as detailed above with respect to actuation mechanism 200 and jaw drive mechanism 600 (FIGS. 5-7), wherein the distal urging of pressurized fluid deploys knife 60 distally and wherein the proximal urging or return of pressurized fluid (under the bias of biasing member 740) retracts knife 60 proximally.

With reference to FIGS. 9 and 10, first and second articulation mechanisms 800, 900 and the actuation thereof via actuation mechanisms 400, 500, respectively, for articulation of end effector assembly 40 are described. As noted above, first articulation mechanism 800, in response to actuation via actuation mechanism 400, may be configured to selectively actuate a first pair of diagonally-opposed articulation cables 38 in opposite directions with equal magnitude, while second articulation mechanism 900, in response to actuation via actuation mechanism 500, may be configured to selectively actuate the other pair of diagonally-opposed articulation cables 38 in opposite directions with equal magnitude. However, other configurations are also contemplated such as, for example, wherein separate articulation assemblies and/or actuation mechanisms are provided for each articulation cable 38, greater than or less than four (4) articulation cables 38 are provided, other pair or groups of articulation cables 38 are coupled for equal and opposite movement, etc.

With particular reference to FIG. 9, as noted above, in response to rotational input provided by robotic surgical system 1000 (FIG. 4) to input coupler 130, pressurized fluid is urged distally through fluid line 238 or pulled (or allowed to return) proximally through fluid line 238 of actuation mechanism 400, depending upon the direction of the rotational input.

Articulation mechanism 800 may be disposed within housing 20 (FIG. 1), within proximal section 34 of shaft 30 (see FIG. 1), partially within each, or otherwise positioned proximally of articulation section 36 of shaft 30 (see FIG. 1). Articulation mechanism 800 may include a first articulation sub-assembly 802 and a second articulation sub-assembly 804. First and second articulation sub-assemblies 802, 804 are both coupled to fluid line 238 of actuation mechanism 400 and are similar to one another except that first and second articulation sub-assemblies 802, 804 are oppositely arranged relative to one another. As a result, and as detailed below, upon actuation of actuation mechanism 400, first and second articulation sub-assemblies 802, 804 manipulate the corresponding articulation cables 38, e.g., the first diagonally-opposing pair of articulation cables 38, in opposite directions with equal magnitude. As an alternative to two sub-assemblies 802, 804, actuation mechanism 400 may alternatively include a single assembly such as detailed below with respect to FIG. 11 wherein the output thereof is modified, e.g., via gears, pulleys, etc., such that, upon actuation, the corresponding articulation cables 38, e.g., the first diagonally-opposing pair of articulation cables 38, are actuated in opposite directions with equal magnitude.

Each articulation sub-assembly 802, 804 includes a fluid vessel 810, a piston head 820 sealingly and slidably disposed within the respective fluid vessel 810 to divide the respective fluid vessel 810 into a fluid-receiving portion 812 and an opposing portion 814, a fluid port 830 fluidly coupling the corresponding fluid-receiving portion 812 with fluid line 238 of piston assembly 230 of actuation mechanism 400, and a biasing member 840, e.g., a coil spring, disposed within the opposing portion 814 and configured to bias the corresponding piston head 820 towards fluid-receiving portion 812 of the corresponding fluid vessel 810. Articulation sub-assemblies 802, 804 are oppositely-oriented relative to one another. A proximal portion of each of the two articulation cables 38 of the first diagonally-opposed pair is coupled to one of the piston heads 820 such that translation of piston heads 820 within fluid vessels 810 likewise translates articulation cables 38. Articulation cables 38 may include substantially rigid support rods 39 disposed thereabout or engaged thereto and extending proximally from articulation section 36 of shaft 30 (see FIG. 1) to provide structural support and rigidity to cables 38, although other configurations are also contemplated.

As a result of the above-detailed configuration, in response to a rotational input to input coupler 130 provided by robotic surgical system 1000 (FIG. 4), piston head 234 is translated within fluid vessel 232 in the first direction to urge pressurized fluid from fluid vessel 232 through fluid line 238 and fluid ports 830 into fluid-receiving portions 812 of fluid vessels 810, whereby the pressurized fluid urges piston heads 820 towards opposing portions 814 of fluid vessels 810 and against the bias of biasing members 840. This translation of piston heads 822, 842, in turn, translates articulation cables 38 in opposite directions (due to the opposite orientation of articulation sub-assemblies 802, 804) and with equal magnitude.

On the other hand, where an opposite rotational input to input coupler 130 is provided by robotic surgical system 1000 (FIG. 4), piston head 234 is translated within fluid vessel 232 in a second opposite direction to draw or allow pressurized fluid to return to fluid vessel 232 such that piston heads 820, under the bias of biasing members 840, are translated in a second direction to translate articulation cables 38 in opposite directions (due to the opposite orientation of articulation sub-assemblies 802, 804) and with equal magnitude.

Referring to FIG. 10, articulation mechanism 900 and actuation mechanism 500 are configured similar to articulation mechanism 800 and actuation mechanism 400 detailed above with respect to FIG. 9, except that articulation mechanism 900 and actuation mechanism 500 operably couple input coupler 140 with the second pair of diagonally opposed articulation cables 38 such that, in response to a rotational input to input coupler 140 provided by robotic surgical system 1000 (FIG. 4), the articulation cables 38 of the second diagonally opposed pair are translated in opposite directions and with equal magnitude.

Turning to FIG. 11, with respect to articulation mechanism 800 (FIG. 9) and/or articulation mechanism 900 (FIG. 10), as an alternative to providing two oppositely-oriented sub-assemblies in each articulation mechanism, an articulation mechanism(s) 1100 may be provided to replace articulation mechanism 800 (FIG. 9) and/or articulation mechanism 900 (FIG. 10). Articulation mechanism 1100 includes a single assembly coupled to the corresponding actuation mechanism 400 or 500 (see also FIGS. 9 and 10, respectively). Articulation mechanism 1100, more specifically, includes a fluid vessel 1110, a piston head 1120 sealingly and slidably disposed within the fluid vessel 1110 to divide the fluid vessel 1110 into a fluid-receiving portion and an opposing portion, a fluid port 1130 fluidly coupling the fluid-receiving portion of the fluid vessel 1110 with the fluid line 238 of the actuation mechanism 400, 500, and a biasing member 1140, e.g., a coil spring, disposed within the opposing portion of the fluid vessel 1110 and configured to bias the piston head 1120 towards the fluid-receiving portion of the fluid vessel 1110. A proximal portion of one of the articulation cables 38 of the diagonally-opposed pair is coupled to piston head 1120, e.g., positively coupled such as, for example, in direct engagement, such that translation of piston head 1120 within fluid vessel 1110 translates that articulation cable 38 in the same direction. The other articulation cable 38 of the diagonally-opposed pair is coupled to piston head 1120, e.g., negatively coupled such as, for example, via a reversing gear assembly 1180, such that translation of piston head 1120 within fluid vessel 1110 translates that articulation cable 38 in the opposite direction. The operation of articulation mechanism 1100, in response to actuation of the coupled actuation mechanism 400 or 500, is similar as detailed above and provides opposite direction and equal magnitude translation of the diagonally opposed articulation cables 38.

With reference again to FIGS. 9 and 10, appropriate inputs to input couplers 130 and/or 140 may be provided to achieve any suitable combination of yaw and/or pitch articulation. For example, and without limitation, where similar inputs are provided to both input couplers 130 and 140, pitch articulation may be effected in either direction based upon the direction of the rotational input. As another example without limitation, where opposite inputs are provided to input couplers 130, 140, yaw articulation may be effected in either direction based upon the directions of the opposite rotational input. Various other inputs may be provided, as noted above, to achieve any suitable combination of yaw and/or pitch articulation.

Continuing with reference to FIGS. 9 and 10, in aspects, rather than providing biasing members to bias the piston heads towards the fluid-receiving portions of the corresponding fluid vessels, the two fluid vessels can be operably coupled to one another to each provide fluid-pressure-based bias to the piston head of the other fluid vessel, thus providing the bias detailed above. With additional reference to FIG. 11, alternatively, a dual-acting piston and fluid vessel (with ports on opposite sides of the piston) may be utilized instead of two separate fluid vessels or a one-port vessel, to achieve the functions detailed above. Regardless of the number and configuration of fluid vessels utilized, the fluid vessel may be sized and/or an appropriate amount of fluid (in volume or via other suitable metric) utilized to maintain a pre-tension on the articulation cables, thus allowing for more precise articulation and helping to hold the position of the end effector assembly when articulation is not actively ongoing. Any or all of the above may likewise apply to jaw drive (see FIGS. 5 and 6) and/or knife deployment (see FIGS. 5, 7, and 8).

Referring generally to FIGS. 5-11, in aspects, the pistons and fluid cylinders may be configured (e.g., in diameter, volume, length, etc.) and/or the amount of fluid (in volume or via other suitable metric) utilized may be selected to provide a desired mechanical advantage (or disadvantage) suitable for the particular actuation desired.

It will be understood that various modifications may be made to the aspects and features disclosed herein. Therefore, the above description should not be construed as limiting, but merely as exemplifications of various aspects and features. Those skilled in the art will envision other modifications within the scope and spirit of the claims appended thereto.

What is claimed is:

1. An actuation assembly of a surgical instrument, comprising:
an actuation mechanism including a piston assembly configured such that positive actuation of the piston assembly urges pressurized fluid from the piston assembly along a fluid line and such that negative actuation of the piston assembly allows the pressurized fluid to return to the piston assembly from the fluid line; and
first and second output mechanisms, each of the first and second output mechanisms including:
a fluid vessel defining a fluid port in fluid communication with the fluid line;
a piston sealingly and slidably disposed within the fluid vessel; and
a driver coupled to the piston,
wherein, the first and second output mechanism are coupled to the actuation mechanism such that:
in response to positive actuation of the piston assembly, the pressurized fluid flows into the fluid ports of the fluid vessels of the first and second output mechanisms to move the pistons of the first and second output mechanisms in a first direction and in a second, opposite direction, respectively, to thereby translate the drivers of the first and second output mechanisms in the first and second directions, respectively, and
in response to negative actuation of the piston assembly, the pressurized fluid exits the fluid ports of the fluid vessels of the first and second output mechanisms to allow the pistons of the first and second output mechanisms to move in the second and first directions, respectively, to thereby translate the drivers of the first and second output mechanisms in the second and first directions, respectively.

2. The actuation assembly according to claim 1, wherein the actuation mechanism further includes a lead screw assembly configured to receive a rotational input and to convert the rotational input into longitudinal motion, the lead screw assembly coupled to the piston assembly such that forward longitudinal motion produced by the lead screw assembly positively actuates the piston assembly and such that reverse longitudinal motion produced by the lead screw assembly negatively actuates the piston assembly.

3. The actuation assembly according to claim 2, wherein the lead screw assembly is configured to receive the rotational input from an input coupler configured to connect to a robotic surgical system.

4. The actuation assembly according to claim 3, wherein the lead screw assembly includes a lead screw coupled with the input coupler such that rotation of the input coupler rotates the lead screw in a similar manner, and a nut threadingly engaged about the lead screw such that rotation of the lead screw translates the nut along the lead screw to provide the longitudinal motion.

5. The actuation assembly according to claim 1, wherein at least a portion of the fluid line is flexible to permit articulation thereof to enable articulation of the output mechanism relative to the actuation mechanism.

6. The actuation assembly according to claim 1, further comprising a flexible cable extending through the fluid line and coupled between a piston of the piston assembly and the piston of the output mechanism.

7. The actuation assembly according to claim 1, wherein the driver of at least one of the first or second output mechanisms is or is connected to an articulation cable configured to articulate an end effector assembly relative to a housing.

8. The actuation assembly according to claim 1, wherein the drivers of the first and second output mechanisms are or are connected to respective first and second articulation cables configured to articulate an end effector assembly relative to a housing.

9. A surgical instrument configured for attachment to a robotic surgical system, the surgical instrument comprising:
a housing;
a shaft extending distally from the housing, the shaft including an articulation section;
an end effector assembly extending distally from the shaft such that the articulation section of the shaft is disposed between the housing and the end effector assembly; and
the actuation assembly according to claim 1.

10. The actuation assembly according to claim 1, wherein the pistons of the first and second output mechanisms are oppositely oriented relative to one another.

11. The actuation assembly according to claim 1, wherein the fluid ports the first and second output mechanisms are disposed at opposite end portions of the fluid vessels of the first and second output mechanisms, respectively.

12. An actuation assembly of a surgical instrument, comprising:
an actuation mechanism including a piston assembly configured such that positive actuation of the piston assembly urges pressurized fluid from the piston assembly along a fluid line and such that negative actuation of the piston assembly allows the pressurized fluid to return to the piston assembly from the fluid line; and
an output mechanisms including:
a fluid vessel defining a fluid port in fluid communication with the fluid line;
a piston sealingly and slidably disposed within the fluid vessel; and
first and second drivers coupled to the piston,
wherein, in response to positive actuation of the piston assembly, the pressurized fluid flows into the fluid port of the fluid vessel to move the piston in a first direction,
wherein, in response to negative actuation of the piston assembly, the pressurized fluid exits the fluid port of the fluid vessel to allow the piston to move in the second direction, and
wherein the first and second drivers are operably coupled to the piston oppositely of one another such that the first and second drivers are moved in the first and second directions, respectively, when the piston is moved in the first direction and such that the first and second drivers are moved in the second and first directions, respectively, when the piston is moved in the second direction.

13. The actuation assembly according to claim 12, wherein the actuation mechanism further includes a lead screw assembly configured to receive a rotational input and to convert the rotational input into longitudinal motion, the lead screw assembly coupled to the piston assembly such that forward longitudinal motion produced by the lead screw assembly positively actuates the piston assembly and such that reverse longitudinal motion produced by the lead screw assembly negatively actuates the piston assembly.

14. The actuation assembly according to claim 13, wherein the lead screw assembly is configured to receive the rotational input from an input coupler configured to connect to a robotic surgical system.

15. The actuation assembly according to claim 14, wherein the lead screw assembly includes a lead screw coupled with the input coupler such that rotation of the input coupler rotates the lead screw in a similar manner, and a nut threadingly engaged about the lead screw such that rotation of the lead screw translates the nut along the lead screw to provide the longitudinal motion.

16. The actuation assembly according to claim 12, wherein at least a portion of the fluid line is flexible to permit articulation thereof to enable articulation of the output mechanism relative to the actuation mechanism.

17. The actuation assembly according to claim 12, further comprising a flexible cable extending through the fluid line and coupled between a piston of the piston assembly and the piston of the output mechanism.

18. The actuation assembly according to claim 12, wherein at least one of the first or second drivers is or is connected to an articulation cable configured to articulate an end effector assembly relative to a housing.

19. The actuation assembly according to claim 12, wherein the first and second drivers are or are connected to respective first and second articulation cables configured to articulate an end effector assembly relative to a housing.

20. A surgical instrument configured for attachment to a robotic surgical system, the surgical instrument comprising:
a housing;
a shaft extending distally from the housing, the shaft including an articulation section;
an end effector assembly extending distally from the shaft such that the articulation section of the shaft is disposed between the housing and the end effector assembly; and
the actuation assembly according to claim 12.
